(19) United States
(12) Patent Application Publication
(10) Pub. No.: US 2008/0255549 A1
(43) Pub. Date: Oct. 16, 2008

(54) PHOTODYNAMIC THERAPY DEVICE

(75) Inventors: Andreas Rose, San Marcos, CA (US); Guenter Herr, Ehringshausen (DE); Kyle Johnston, Sammamish, WA (US)

Correspondence Address: DOBRUSIN & THENNISCH PC, 29 W LAWRENCE ST, SUITE 210, PONTIAC, MI, 48342, US

(73) Assignee: Ondine International, Ltd.

(21) Appl. No.: 11/868,677

(22) Filed: Oct. 8, 2007

Related U.S. Application Data
(60) Provisional application No. 60/829,912, filed on Oct. 18, 2006.

(51) Int. Cl. A61B 18/18

(52) U.S. Cl. 606/15

(57) ABSTRACT

The invention described herein is an apparatus for performing photodynamic therapy comprising: a waveguide, a light source in communication with one end of the waveguide, a photodynamic tip in communication with another end of the waveguide wherein the photodynamic tip is designed to be securely but removably attached to a scaling tip of a scaler and the apparatus delivers light in a desired illumination pattern and in at least one predetermined wavelength to activate a photosensitizing composition for the killing of microbes. The invention also includes a method for using the apparatus in conjunction with a scaler to perform sonophotodynamic therapy and a method for making the apparatus.

### CLAIM OF BENEFIT OF FILING DATE

This application claims the benefit of the filing date of U.S. Provisional Application Ser. No. 60/829,912 filed on Oct. 18, 2006, and incorporated herein by reference for all purposes.

### TECHNICAL FIELD

The present invention relates to a medical device for performing photodynamic therapy upon tissue of an organism. More particularly, the invention is a medical device that combines a conventional sonic delivery apparatus with a light delivery apparatus to deliver electromagnetic radiation and sonic energy to an area under treatment.

### BACKGROUND OF THE INVENTION

Photodynamic therapy (“PDT”) has been used to treat various maladies and diseases. PDT often involves the use of a photosensitizing agent that is activated by electromagnetic radiation (e.g., light such as laser light). PDT for killing microbes in the oral cavity, also sometime known as photodisinfection, was disclosed by Wilson, et al. in U.S. Pat. No. 5,611,793 and European Patent No. EP 0637976B2. Recent inventions have shown that PDT or photodisinfection can be combined with sonic energy (hereinafter referred to as “sonophotodynamic therapy”) to achieve a significant increase in disinfection effectiveness, especially for the treatment of periodontal and dental diseases. See U.S. patent application Ser. Nos. 11/144,280 (Pub. No. 2006-0019220) and 11/144,433 (Pub. No. 2006/0010220), and PCT/US 2005/019707 (Pub. No. WO06/022970), all filed on Jun. 3, 2005.

Dental scaling is the use of sonic energy to clean patients' gum and teeth. Dental scaling is performed on a patient generally twice a year and on patients with periodontal diseases several times a year, in some cases every three months or more frequently. Dental scaling is often performed with a conventional ultrasonic or sonic scaler (collectively thereinafter referred to as “scaler”. See Position Paper: Sonic and Ultrasonic Scalers in Periodontics, J. Periodontal 2000:1792-1801). A scaler generates sonic energy (e.g., vibrations) in a fluid (e.g., water, saline or the like) that remove subgingival plaque, calculus and biofilm from the gum tissues. The vibrations cause cavitation exerting high shear forces directly on the fluid, the calculus, and the plaque surrounding or within the gum tissue, resulting in the detachment of such calculus, plaque and associated biofilm from the gum tissues. The principles of scalers are well described in the patent literature. See U.S. Pat. Nos. 2,990,616; 3,089,790; 3,703,037; 3,990,452; 4,283,174; 4,804,364; and 6,619,957. Scalers are widely used and can be found in most dental offices.

### SUMMARY OF THE INVENTION

The present invention provides an apparatus and a method by which a scaler can be used with little or no modification to conduct sonophotodynamic therapy, thereby bringing the benefits of sonophotodynamic therapy to many users without the need for replacing existing scalers.

In one embodiment, the present invention is an apparatus for performing photodynamic therapy comprising: a waveguide, a light source in communication with one end of the waveguide, a photodynamic tip in communication with another end of the waveguide wherein the photodynamic tip is designed to be securely but removably attached to a scaling tip of a scaler and the apparatus delivers light in a desired illumination pattern and in at least one predetermined wavelength to activate a photosensitizing composition for killing of microbes.

In another embodiment, the present invention is a method for performing sonophotodynamic therapy comprising: providing sonic energy to desired treatment area using a scaler; providing a photosensitizing composition to the desired treatment area; providing light in a desired illumination pattern and in at least one predetermined wavelength to activate the photosensitizing composition using the above-described apparatus.

In yet another embodiment, the present invention is a method for making an apparatus for performing photodynamic therapy comprising providing a waveguide, a light source, and a photodynamic tip having a scaling tip feature for secured but removable attachment to a scaling tip of a ultrasonic scaler; attaching one end of the waveguide to the photodynamic tip; attaching other end of the waveguide to the light source, wherein the apparatus can deliver light in a desired illumination pattern and in at least one predetermined wavelength to activate a photosensitizing composition for the killing of microbes.

A better understanding of the invention will be had upon review of the follow detailed description, which is to be read in conjunction with the accompanying drawings.

### BRIEF DESCRIPTION OF THE DRAWINGS

In the drawings, like reference numerals and letters refer to like parts throughout the various views, unless indicated otherwise:

FIG. 1 is a pictorial view of a scaler;

FIG. 2 is a pictorial view of an apparatus for photodynamic therapy constructed in accordance with an embodiment of the invention;

FIG. 3 is a pictorial view of the apparatus in FIG. 2 attached to the handset of the scaler shown in FIG. 1;

FIG. 4 is a side cross-sectional view of the photodynamic tip shown in FIG. 2;

FIG. 5 is a side cross-section view of another embodiment of the photodynamic tip in accordance with an embodiment of the invention;

FIG. 6 is a pictorial view of an example of a single use means in accordance with an embodiment of the invention;

FIG. 7 is a side pictorial view of the example of a single use means shown in FIG. 6; and

FIG. 8 is a pictorial view of an apparatus for photodynamic therapy constructed in accordance with an embodiment of the invention.

### DESCRIPTION OF THE PREFERRED EMBODIMENT

The present invention is predicated upon providing an apparatus that can be used with a scaler to perform sonophotodynamic therapy upon tissue of an organism. Generally, it is contemplated that the present invention may be employed to perform sonophotodynamic therapy upon any tissue of any organism alive or dead and/or upon objects such as denture or other prosthetics and should not be limited to performing therapy on any particular tissue, organism or other object unless otherwise specifically recited. The apparatus has been found to be particularly useful, however, for performing photodynamic therapy and/or sonophotodynamic therapy (i.e., used in conjunction with a scaler) upon tissue within the oral cavities of humans. The present invention allows photodynamic therapy and/or sonophotodynamic therapy to be performed during regular dental scaling treatment using an existing scaler thereby saving time and costs.

### I. Definitions

The following terms are intended to have the following general meanings as they are used herein.

1. Microbes: any and all disease-related microbes such as virus, fungus, and bacteria including Gram-negative organisms, Gram-positive organisms or the like.

2. Light: light at any wavelengths that can be absorbed by a photosensitizing composition. Such wavelengths include wavelengths selected from the continuous electromagnetic spectrum such as ultraviolet (“UV”), visible, the infrared (near, mid and far), etc. The wavelengths are generally preferably between about 160 nm to 1600 nm, more preferably between 400 nm to 800 nm, most preferably between about 500 nm to 850 nm although the wavelengths may vary depending upon the particular photosensitizing compound used and the light intensity. The light may be produced by any suitable art-disclosed light emitting devices such as lasers, light emitting diodes (“LEDs”), arc lamps, incandescent sources, fluorescent sources, gas discharge tubes, thermal sources, light amplifiers or the like.

3. Photosensitizing composition: a composition comprising at least one suitable art-disclosed photosensitizer. Arianor steel blue, toluidine blue 0, crystal violet, methylene blue and its derivatives, azure blue cert, azure B chloride, azure 2, azure A chloride, azure B tetrafluoroborate, thionin, azure A eosinate, azure B eosinate, azure mix sicc., azure II eosinate, haematoporphyrin HCI, haematoporphyrin ester, aluminium disulphonated phthalocyanine are examples of suitable photosensitizers. Porphyrins, pyrroles, tetrapyrrolic compounds, expanded pyrrolic macrocycles, and their respective derivatives are further examples of suitable photosensitizers. Photofrin® manufactured by QLT PhotoTherapeutics Inc., Vancouver, B.C., Canada is yet another example of a suitable photosensitizer. Other exemplary photosensitizers may be found in U.S. Pat. Nos. 5,611,793 and 6,693,093. U.S. Pat. No. 6,693,093 is hereby incorporated by reference. The photosensitizers mentioned above are examples are not intended to limit the scope of the present invention in any way.

4. Sonic energy: ultrasound, sonic waves or energy produced by a sonic or ultrasonic device (e.g., ultrasonic scaler, sonic scaler, or the like).

### II. Conventional Scaler

FIG. 1 illustrates an exemplary scaler 10 comprising a handset 12 having a scaling tip 14; an irrigation channel 16 connected to a fluid source 18 such that when activated by a controller (not shown), fluid (e.g., water, saline, combinations thereof or other fluids) is delivered from the fluid source 18 through the irrigation channel 16 to the desired treatment area; a piezoelectric or ultrasonic driver mechanism 20 that is in electrical communication with an electrical energy source 22 wherein when activated by a controller (not shown), the driver mechanism 20 converts energy from the electric energy source 22 into ultrasonic vibrations deliverable to the desired treatment area by the scaling tip 14.

### III. Apparatus of the Present Invention

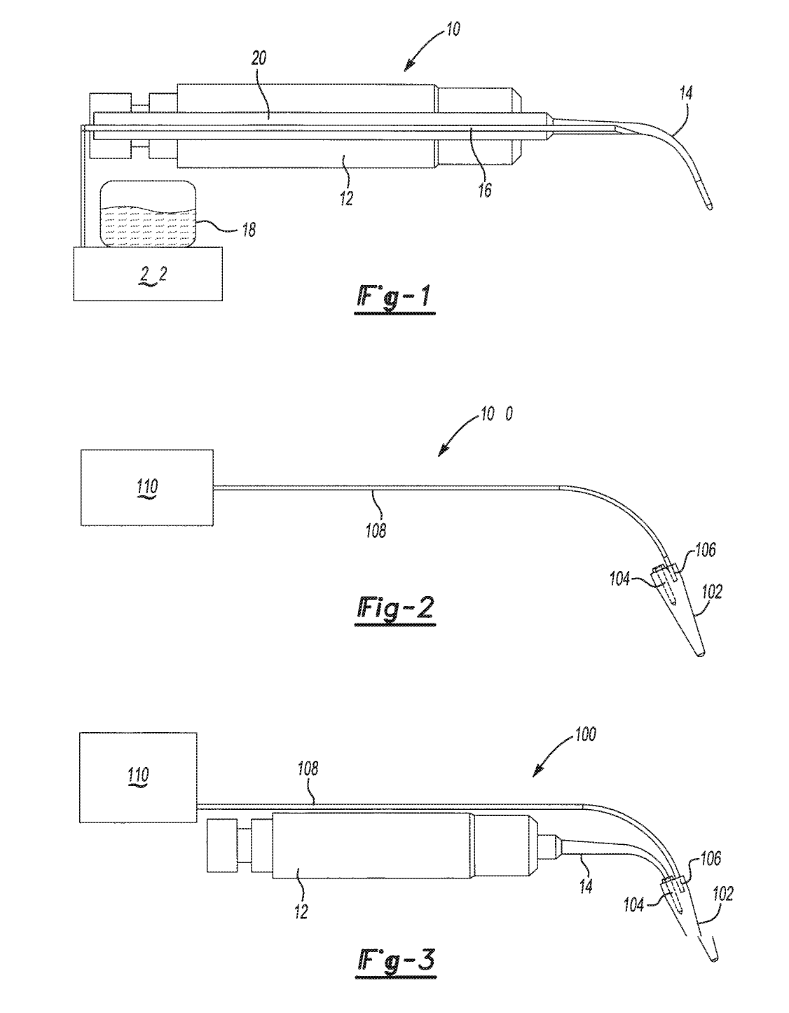

FIG. 2 illustrates an exemplary apparatus 100 of the present invention for performing sonophotodynamic therapy comprising a photodynamic tip 102 having a scaling tip feature 104 and a waveguide feature 106, a waveguide 108 in communication with the photodynamic tip 102, a light source 110 in communication with the waveguide 108. Light is communicated from the light source 110 along the waveguide 108, which guides the light to the photodynamic tip 102 where it is emitted in a desired illumination pattern and at least one predetermined wavelength to activate a photosensitizing composition for killing of microbes that are located on and/or within tissue of an organism.

The waveguide feature 106 accepts one end of a waveguide 108 allowing the waveguide 108 to be attached to and in communication with the photodynamic tip 102. The scaling tip feature 104 is designed to accept the scaling tip 14 and provide secured but removable attachment between the photodynamic tip 102 and the scaling tip 14 as shown in FIG. 3.

FIG. 4 provides a more detailed view of the photodynamic tip 102. The scaling tip feature 104 is a pocket that accepts the scaling tip 14. In one example, the pocket is about 1 mm diameter on one end and tapers to about 0.5 mm diameter on the other end allowing the scaling tip feature 104 to securely fit approximately the last 5 mm of the scaling tip 14. The fit is tight enough so that a combination of vacuum pressure and friction serve to hold the photodynamic tip 102 securely in place to the scaling tip 14 without the need of other retention mechanisms. Alternatively, the scaling tip feature 104 (e.g., the pocket or the like) may also have additional retention features. For example and as illustrated in FIG. 5, ribs 112 are formed within the scaling tip feature 104. The ribs 112 are formed so they deflect slightly when the scaling tip 14 in inserted in the scaling tip feature 104, providing a spring pressure to clamp the photodynamic tip 102 onto the scaling tip 14. In addition the pocket geometry discussed above, it would be possible to design other retention techniques for attaching the photodynamic tip 102 to the vibrating scaling tip 14. One such technique would be a simple split in the photodynamic tip's 102 body running part way down the outside edge of the pocket (the scaling tip feature 104). This would allow the body to flex slightly to open the split allowing the scaling tip 14 to be placed or forced into the pocket, providing clamping pressure to retain the photodynamic tip 102 in place with the scaling tip 14. It is contemplated and within the scope of this invention that the skilled artisan may be able to employ a wide variety of other pocket geometries and retention features and to temporarily attach the photodynamic tip 102 to the scaling tip 14 using an adhesive.

The waveguide 108 can be retained by the waveguide feature 106 using suitable art-disclosed means such as friction, ribs, mechanical clamping features, threads, ultrasonic staking, adhesive or the like. For example and referring to FIG. 4, the waveguide feature 106 can for example be a pocket that directly accepts a portion (e.g., about 2 mm or the like) of the waveguide 108. The pocket can have a tapered entry diameter 114 that makes it easier to insert the waveguide 108. If adhesive is used to further secure the attachment between the waveguide 108 and the photodynamic tip 102, the pocket can also acts as a reservoir for the adhesive while it cures.

Referring to FIG. 4, the waveguide feature 106 may have a flat interface 116 allowing light from the waveguide 108 to transmit directly into the body of the photodynamic tip 102 without changing its intensity distribution. However, it may be desirable to change the intensity distribution of the light in order to optimize the emission pattern into the treatment area. As shown in FIG. 5, this can be achieved by providing the waveguide feature 106 with a shaped interface 118. The hemispherical concave surface of the shaped interface 118 will act as a negative lens and will spread the light from the waveguide 108 out into higher propagating angles. The light distribution modifying features could be formed to spread the light out or to narrow the pattern, depending on the output emission pattern desired. Without limitation, some useful features for the shaped interface 118 are concave and convex curved surfaces, concave and convex cones, prismatic facets, holographic patterns, diffractive patterns, and Fresnel type lenses. The waveguide's 108 end that interfaces with the shaped interface 118 may also be modified by such features to achieve redistribution of the light.

The photodynamic tip 102 is constructed of substantially transparent material to allow the light to efficiently propagate through its body. The photodynamic tip 102 can be formed from a wide variety of materials. For example, plastic (e.g., acrylic, polycarbonate, polystyrene, or the like), resin (i.e., an epoxy or the like), glass or the like. Using a clear plastic (e.g., polycarbonate, acrylic, or the like) allows the photodynamic tip 102 to be formed by a molding process, resulting in high quality parts with a very low parts cost.

It may be preferred that light delivered to the treatment area from the photodynamic tip 102 has low light loss and an optimal distribution pattern without any especially bright or dim spots. The desired treatment area is generally in front of the photodynamic tip 102, not behind it, and therefore light heading back towards the waveguide 108 will be wasted. Therefore, efficient illumination requires that the majority of the light emitted from the photodynamic tip 102 be heading towards its distal end away from the waveguide 108.

FIG. 4 illustrates an example of the photodynamic tip 102 that provides efficient illumination. In this example, the base cylinder section of the photodynamic tip 102 has a diameter of about 3.25 mm and extends about 2.3 mm long. The taper section of the photodynamic tip 102 then extends about 9.25 mm long and reduces to a diameter of about 1 mm. The distal end of the photodynamic tip 102 has a radius to protect the patient and further distribute the light emitted from the very end. The taper section of the photodynamic tip 102 serves several purposes. It reduces the size of the distal tip so it can be inserted into treatment areas with limited access. Also, as the arrows shown in FIG. 4, the taper section changes the light distribution with each internal reflection, helping to ensure a constant illumination pattern out of the photodynamic tip 102. Some of the high angle light rays out of the waveguide 108 encounter the exterior wall of the photodynamic tip 102 below the critical angle for total internal reflection and are therefore coupled out of the photodynamic tip 102 and refracted towards the treatment area. Some of the lower angle rays are at an angle greater than the critical angle and will therefore be internally reflected. However, due to the taper section of the photodynamic tip 102, each subsequent time a light ray encounters the exterior wall it will be at a lower incident angle relative to the surface normal than the previous encounter. As the light progresses down the body of the photodynamic tip 102, more and more of the light falls below the critical angle and is emitted with a propagation angle that directs it towards the treatment area. Thus, the taper section of the photodynamic tip 102 helps efficiently ensure a constant illumination pattern out of the photodynamic tip 102.

In addition to the example shown in FIG. 4, a wide variety of alternate body dimensions for the photodynamic tip 102 can be utilized depending upon the desired application(s) and the treatment area(s) and the accessibility (e.g., opening or the like) to such treatment area(s). A skilled person in the arts would have to take into account the material choice to make the trade off between length of the body and the amount of taper provided to ensure that the final photodynamic tip 102 design has the required rigidity and strength. Once a mechanical form that fits the application treatment is determined, there remains the issue of ensuring the light is emitted in an appropriate manner. Typically, the longer the body and the smaller the taper section, the more likely the light will guide through the photodynamic tip 102 and the less likely that light will be emitted, except at the very distal end of the photodynamic tip 102.

Surface finish of the photodynamic tip 102 can also contribute to the light distribution and pattern. For example, in one embodiment of the photodynamic tip 102, its taper section has a random rough surface finish (e.g., about 30 um rough surface features) that fills about 25% of the clear area of the surface of the photodynamic tip 102. This causes about 25% of the light rays encountering a rough patch on the surface of the photodynamic tip 102 are scattered out of its body regardless of their incident angle. In this fashion, the surface finish helps ensure that all the light leaks out of the photodynamic tip 102 in a uniform manner.

In addition to random rough surfaces, surface modification features can be utilized to couple out light and still be within the scope of this invention. Without limitation, these include concave and convex dimples, concave and convex prismatic facets and annular features. As illustrated in FIG. 5, a preferred series of annular grooves 120 extending around the circumference of the photodynamic tip 102 are provided. As shown, the depth, spacing and shape of the grooves 120 are designed such that the majority of the light rays (some of which are shown as arrows in FIG. 5) coming from the waveguide 108 encounter a surface at an angle that is below the critical angle and thus the light rays are coupled out of the photodynamic tip 102 at the location and angle of the designer's choosing. This embodiment allows the majority of the light to be efficiently coupled out without any internal guiding at all. The surface modification features can also be combined with surface finish variation to further tailor the light emissions.

There are other techniques that could be used to modify the light emission pattern from the apparatus that would still be in the scope of this invention. For instance, another way to get a uniform output would be to include a material in the bulk of the photodynamic tip's 102 body material that causes internal scattering as the light propagates towards the distal end of the photodynamic tip 102. Without limitation, this material could be a pigment type material such as Titanium Dioxide or material with a different refractive index, such as glass micro spheres or even hollow plastic micro spheres.

The waveguide 108 can be any type of optical fiber including various glass fibers, liquid core fibers, and hard clad plastic and glass fibers. It is contemplated and within the scope of the present invention that the waveguide 108 may include a single optical fiber or multiple optical fibers. Additionally, any type of fiber termination can be utilized on either end of the fiber, including various types of standard ferrules and connector adapters (i.e. ST, SMA, etc.). Custom ferrules can also be utilized, including simple metal tube ferrule and ferrules cast onto the end of the fiber. With a fiber ferrule, the waveguide feature 106 would need to have a larger diameter. Many techniques could be used to retain the fiber ferrule within the waveguide feature 106, including without limitation, adhesives, friction, mechanical clamping features, threads or the like.

As a practical method of ensuring sterility, it may be desirable to construct the apparatus 100 with low cost materials allowing disposability after single use. However, the apparatus can also be created with materials (e.g., glass or the like for the photodynamic tip 102 and/or the waveguide 108) that can withstand standard sterilization techniques such as autoclaving.

In one embodiment, the waveguide 108 is an inexpensive plastic optical fiber with its end bonded into the waveguide feature 106 resulting in a permanent attachment between the photodynamic tip 102 and the waveguide 108. The waveguide 108 can have a flat polished end, forming a low loss optical connection. When the waveguide 108 is permanently attached to the photodynamic tip 102, protection from exposure of harmful levels of optical radiation may be provided to the user and patient. This design provides eye safety without the need for extensive additional optical safety procedures and gear.

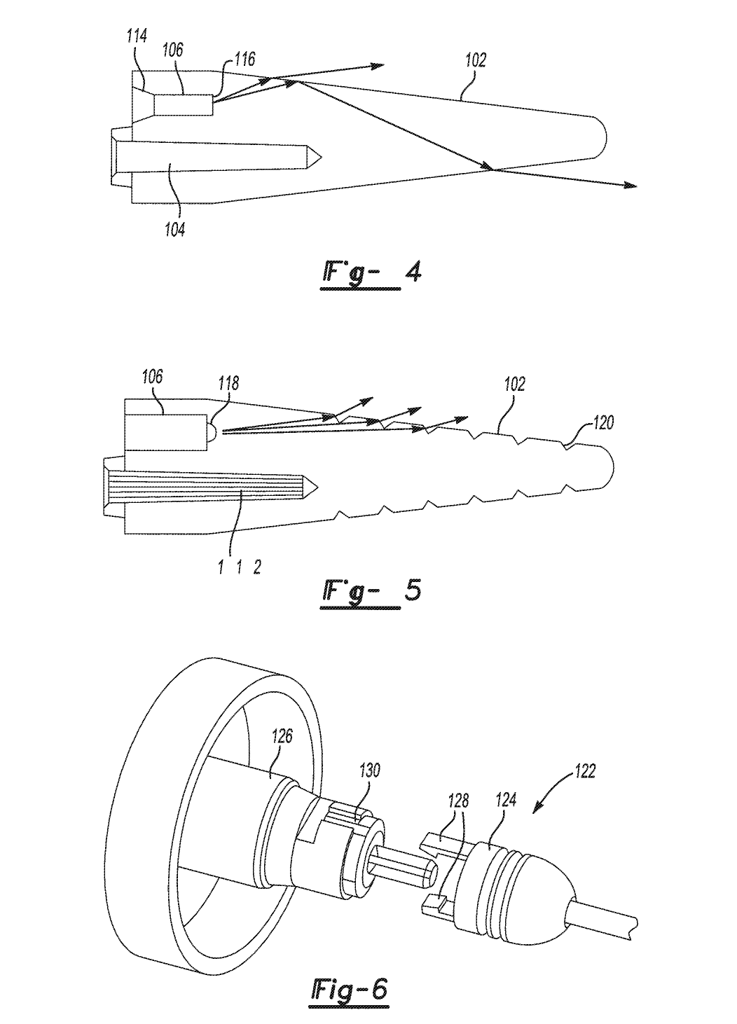

The waveguide 108 can also be constructed in accordance with the invention disclosed in commonly owned U.S. patent application Ser. No. 11/397,768 filed on Apr. 4, 2006 and PCT/US 2006/13380 filed on Apr. 11, 2006. The waveguide 108 may optionally include single use means 122 for the waveguide's communication with the light source 110. These patent applications are hereby incorporated by reference in their entirety. FIGS. 6-7 illustrate examples of the single use means 122 which includes a single use connector 124 (shown as a molded plastic single use optical fiber adapter) attached to proximal end of the waveguide 108 and a connector interface 126 (shown as a ferrule) attached to the light source 110. The single use connector 122 may include retention tang 128 features such that when the single use connector 122 is first engaged with the connector interface 126, the retention tang features 128 spread slightly and engage with the interface feature 130 on the connector interface 126. However, when the single use connector 124 is disconnected or removed form the connector interface 126, the retention tang features 128 are destroyed. In this fashion, the single use connector 122 will not have any reusable retention tang features 128, discouraging subsequent use and provides disposability. This arrangement also allows the connector interface 126 to be exposed and periodically cleaned to maintain low light loss optical connectivity.

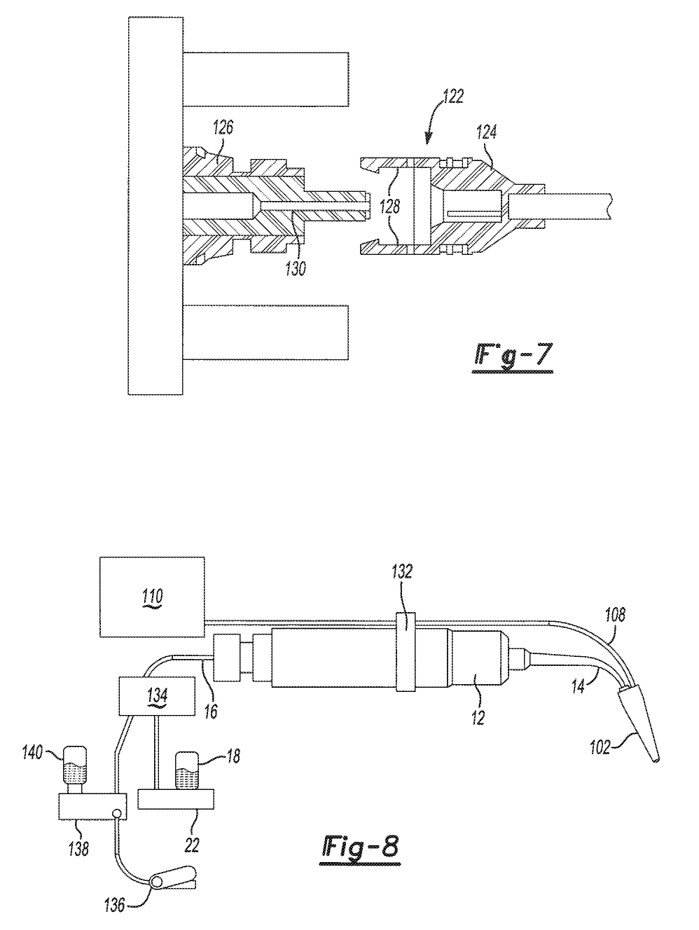

The apparatus 100 may optionally further include a waveguide attachment means 132 to keep the waveguide 108 from getting in the way during treatment. The waveguide attachment means 132 can be any suitable art-disclosed device. For example, the waveguide attachment means 132 can be a simple strip of adhesive tape attaching the waveguide 108 to the handset 12. Another example of the waveguide attachment means 132 is a clip as shown in FIG. 8. The clip is a molded plastic “C” clamp that attaches the waveguide 108 to the handset 12. The waveguide attachment means 132 can be optionally retained onto the waveguide 108 so that the waveguide 108 can be attached to the handset 12 with a simple motion without having to align both the waveguide 108 and the handset 12 before applying the waveguide attachment means 132. It is contemplated that the skilled artisan may be able to employ a variety of alternate structures and a wide variety of material with mild spring force that would serve as the waveguide attachment means 132.

It is contemplated and within the scope of the present invention that a variety of suitable art-disclosed means can be used to deliver the photosensitizing composition to the desired treatment area. For example, the photosensitizing composition can be delivered using a fluid applicator such as syringe, a pipette, or the like.

This fluid applicator can be designed for single use and packaged in a disposable kit that further includes the apparatus 100. Alternatively, the kit can exclude the light source 110 and comprise the fluid applicator, the photodynamic tip 102 connected to the waveguide 108. In this embodiment, it is preferred that the waveguide 108 is attached to the light source 110 (not included in the kit) via the single use means 122. The disposable kits discussed herein may also include the photosensitizing composition, either stored within the fluid applicator or in a separate container.

It is also contemplated and within the scope of the present invention that the photosensitizing composition be delivered using the irrigation channel 16 of the ultrasonic scaler 10. The photosensitizing composition can be delivered to the irrigation channel 16 using various art-disclosed means. For example and as illustrated in FIG. 8, a manifold 134 allowing fluid from multiple sources to be injected into the irrigation channel 16 is provided and a controller 136 (e.g., a hand switch, a foot switch or the like) can be used to activate a pump 138 that draws the photosensitizing composition from a photosensitizing composition source 140 and injects it into the manifold 134 and then to the irrigation channel 16. In this fashion, the existing irrigation channel 106 can be used to deliver the photosensitizing composition at any time during treatment without interruption or requiring the switching out of tools.

It is also within the scope of this invention if a separate fluid tube outside of the scaler 10 is used to deliver the photosensitizing composition to the treatment area. For example, a separate fluid tube connected to a photosensitizing composition source can run parallel with the waveguide 108 and terminate in a location where the photosensitizing composition exiting the distal end of the fluid tube 138 would land in the treatment area.

It is also within the scope of this invention for the apparatus 100 to be used with any art-disclosed suitable manual scaler or periodontal probe (i.e. a scaler or periodontal probe that does not provide any sonic energy) by attaching the photodynamic tip 102 to the tip of the manual scaler or periodontal probe.

### IV. Method of the Present Invention

The present invention provides a method to perform sonophotodynamic therapy comprising: providing sonic energy to desired treatment area using an ultrasonic scaler; providing a photosensitizing composition to said desired treatment area; providing light in a desired illumination pattern and in at least one predetermined wavelength to activate the photosensitizing composition using the apparatus 100. To perform the light step, an operator attaches the photodynamic tip 102 to the scaling tip 106 and activates the light source 110. The providing a photosensitizing composition step must be performed before the providing light step. The providing a photosensitizing composition step can be performed by delivering the photosensitizing composition to the desired treatment area via various art-disclosed means (e.g., a separate applicator, the irrigation channel 16, or the like). The scope of this invention allows for the providing sonic energy step to be performed before, during, and/or after the providing light step.

If desired, it is optional and within the scope of the invention for the photodynamic tip 102 to perform scaling of the treatment area (e.g., gum, tooth and other tissues). Thus, the scaler can be used to perform scaling followed by attaching the waveguide to the scaler with the photodynamic tip such that photodynamic therapy can be performed. Alternatively, the photodynamic tip can be attached to the scaler and can be used for scaling before during or after performing photodynamic therapy.

As yet another alternative, the photodynamic tip can be attached to the scaler and the scaler can be set to a low amplitude for moving (e.g., mixing or stirring) photosensitizing composition during the performance of photodynamic therapy. In such a situation, the scaler is typically operated at an amplitude that is below desired scaling amplitudes. Such an amplitude is typically less than about 10 microns, more typically less than 5 micron and even possibly less than 2 microns. Advantageously, such movement can aid the photodynamic therapy in killing greater amounts of microbes (e.g., bacteria).

The above description is intended to be exemplary in nature only. A person skilled in the art would understand that there are different kinds of materials that could be used to make the apparatus 100 described above. Therefore, the foregoing description is not intended to limit what is considered to be the spirit and scope of the invention. The scope of the invention is to be limited only by the claims that follow, the interpretation of which is to be made in accordance with the standard doctrines of patent claim interpretation.

Unless stated otherwise, dimensions and geometries of the various structures depicted herein are not intended to be restrictive of the invention, and other dimensions or geometries are possible. Plural structural components can be provided by a single integrated structure. Alternatively, a single integrated structure might be divided into separate plural components. In addition, while a feature of the present invention may have been described in the context of only one of the illustrated embodiments, such feature may be combined with one or more other features of other embodiments, for any given application. It will also be appreciated from the above that the fabrication of the unique structures herein and the operation thereof also constitute methods in accordance with the present invention.

1-20. (canceled)

21. An apparatus for performing photodynamic therapy comprising:
a waveguide;
a light source in communication with the waveguide and providing light to the waveguide; and
a photodynamic tip having (i) a first pocket adapted to receive an end of the waveguide and is in communication with the end of the waveguide; (ii) a second pocket adapted to receive a scaling tip of a scaler and to provide a secured but removable attachment to the scaling tip; and (iii) a conical shape comprising of a base cylinder section and a taper section wherein distal end of the taper section is smaller in size compared to remaining portions of the photodynamic tip; wherein (a) the light source delivers light to the photodynamic tip via the waveguide; and (b) the photodynamic tip emits the light in at least one predetermined wavelength for the killing of microbes, and with a propagation angle that directs the light toward the distal end of the taper section.

22. An apparatus as in claim 21 wherein the first pocket further includes a shaped interface that directs the light toward the distal end of the taper section.

23. An apparatus as in claim 22 wherein the shaped interface is selected from a group consisting of concave curved surface, convex curved surface, concave cone, convex cone, prismatic facets, holographic patterns, diffractive patterns, Fresnel type lenses and a combination thereof.

24. An apparatus as in claim 22 wherein the second pocket includes retention features that assist the photodynamic tip in securely but removably attaching to the scaling tip.

25. An apparatus as in claim 24 wherein the retention features are ribs that deflect when the scaling tip is inserted into the scaling tip feature thereby clamping the photodynamic tip to the scaling tip.

26. An apparatus as in claim 21 wherein the scaling tip feature is a split in the photodynamic tip that opens upon flexing of the photodynamic tip.

27. An apparatus as in claim 21 wherein the photodynamic tip is formed by a molding process of a substantially transparent plastic material.

28. An apparatus as in claim 21 further comprising an attachment that attaches the waveguide to the scaler, the attachment being a clamp.

29. An apparatus as in claim 21 wherein the first pocket and the second pocket are the same in that they form one single opening within the photodynamic tip and the end of the waveguide and the scaler tip are both received by the one single opening.

30. An apparatus as in claim 21 wherein the photodynamic tip is tapered from adjacent the pocket that receives the waveguide to a distal end of the scaling tip.

31. An apparatus as in claim 29 wherein the pocket that receives the waveguide includes a shaped interface that narrows a pattern of the light.

32. An apparatus as in claim 21 wherein the photodynamic tip includes surface modification features that direct light into a desired pattern.

33. An apparatus for performing photodynamic therapy comprising:
a waveguide;
a light source in communication with a first end of the waveguide and providing light to the waveguide;
a photodynamic tip in communication with a second end of the waveguide;
a scaler having a scaling tip; and
an attachment that attaches the waveguide to a handset of the scaler; wherein:
the photodynamic tip comprising: (i) a first pocket adapted to receive the second end of the waveguide and is in communication with the second end of the waveguide; (ii) a second pocket adapted to receive the scaling tip of the scaler and to provide a secured but removable attachment to the scaling tip; and (iii) a conical shape comprising of a base cylinder section and a taper section wherein distal end of the taper section is smaller in size compared to remaining portions of the photodynamic tip; wherein (a) the light source delivers light to the photodynamic tip via the waveguide; and (b) the photodynamic tip emits the light in at least one predetermined wavelength for the killing of microbes, and with a propagation angle that directs the light toward the distal end of the taper section.

34. A method of performing photodynamic therapy comprising:
providing a scaler with a scaling tip;
providing a light source in communication with a first end of a waveguide;
providing a photodynamic tip comprising: (i) a first pocket adapted to receive a second end of the waveguide and is in communication with the second end of the waveguide; (ii) a second pocket adapted to receive the scaling tip of the scaler and to provide a secured but removable attachment to the scaling tip; and (iii) a conical shape comprising of a base cylinder section and a taper section wherein distal end of the taper section is smaller in size compared to remaining portions of the photodynamic tip;
attaching the photodynamic tip to the scaler tip via the second pocket; and
applying light from the light source to the photodynamic tip via waveguide resulting in the photodynamic tip emitting the light (a) onto a treatment area in at least one predetermined wavelength for killing of microbes located at the treatment area; and (b) with a propagation angle that directs the light toward the distal end of the taper section.

35. A method as in claim 34 further comprising providing sonic energy to the treatment area using the scaler.

36. A method as in claim 34 further comprising providing a photosensitizing composition to the treatment area prior to the applying light onto the treatment area step.

37. A method as in claim 35 wherein at least a portion of the sonic energy is provided to the photodynamic tip by the scaling tip which causes the photodynamic tip to move (i) at an amplitude of less than 5 microns and (ii) the photosensitizing composition.

38. The method as in claim 34 further comprising attaching the waveguide to a handset of the scaler with an attachment.

39. The method as in claim 34 wherein the first pocket further includes a shaped interface that directs the light toward the distal end of the taper section.

40. The method as in claim 34 wherein the photodynamic tip includes surface modification features that direct light into a desired pattern.
